(19) United States
(12) Patent Application Publication
(10) Pub. No.: US 2023/0245715 A1
(43) Pub. Date: Aug. 3, 2023

(54) METHOD FOR PREDICTING POSSIBILITY OF IMMUNOTHERAPY FOR COLORECTAL CANCER PATIENT

(71) Applicant: Gil Medical Center, Incheon (KR)

(72) Inventors: Jung Ho Kim, Incheon (KR); Seungyoon Nam, Incheon (KR); Jungsuk An, Incheon (KR); Sungjin Park, Incheon (KR)

(73) Assignee: Gil Medical Center, Incheon (KR)

(21) Appl. No.: 18/177,439

(22) Filed: Mar. 2, 2023

Related U.S. Application Data
(63) Continuation-in-part of application No. PCT/KR2021/011804, filed on Sep. 2, 2021.

(30) Foreign Application Priority Data
Sep. 3, 2020 (KR) 10-2020-0112176

(51) Int. Cl. G16B 20/20; G16B 40/30; G16H 50/70

(52) U.S. Cl. CPC G16B 20/20; G16B 40/30; G16H 50/70

(57) ABSTRACT

The present invention relates to a method for predicting the possibility of immunotherapy for a colorectal cancer patient. Specifically, by using a discriminant of a multiple linear model for determining the applicability of anticancer immunotherapy to obese colorectal cancer patients of the present invention, the patients are classified into two patient groups according to the type of gene mutation, and a patient group with a high immune signature is selected as a group to which immunotherapy can be applied such that it is possible to provide the benefit of providing a new treatment opportunity to obese colorectal cancer patients who have not benefited from the anticancer immunotherapy.

### CROSS REFERENCE TO RELATED APPLICATION

This patent application is a continuation-in-part of PCT/KR2021/011804, filed Sep. 2, 2021, which claims the benefit of priority from Korean Patent Application No. 10-2020-0112176, filed on Sep. 3, 2020. The contents of both patent applications are incorporated herein by reference.

### BACKGROUND OF THE INVENTION

### 1. Field of the Invention

The present invention relates to a method for predicting the possibility of immunotherapy for a colorectal cancer patient.

### 2. Description of the Related Art

Colorectal cancer is a type of cancer that occurs in areas of the large intestine, rectum, and appendix. Globally, 1.4 million cases of colon cancer were diagnosed in 2012, and 694,000 people died of colon cancer. Colorectal cancer is the fourth most common cancer in the United States and the third most common cause of death in the West. Colorectal cancer is a type of cancer caused by the growth of polyps from adenomas in the large intestine. In most cases, polyps grow into benign tumors, but some develop into malignant tumors. Colorectal cancer is mainly detected through colonoscopy.

Colorectal cancer can be divided into three types according to microsatellite instability: high-level microsatellite instability (high-level MSI, MSI-H), low-level microsatellite instability (low-level MSI, MSI-L) and microsatellite stable (MSS). Microsatellite refers to short repetitive DNA sequences scattered in chromosomes, and the length of the microsatellite varies from person to person. Microsatellite instability refers to a difference in length due to the insertion or deletion of repetitive sequences in the microsatellite within the cancer tissue when comparing the normal tissue and the cancer tissue in the same person. In other words, microsatellite instability is a phenomenon in which the length of a repetitive microsatellite sequence distributed in all genes changes as the accumulation of point mutations of nucleotides accelerates because errors generated during the DNA replication process cannot be corrected due to abnormalities in the DNA mismatch repair system. When the gene repair system is damaged due to microsatellite instability, tumors can develop while reducing the ability to relieve stress caused by chronic inflammation.

On the other hand, anticancer agents for treating colorectal cancer can be classified into first generation chemotherapy agents, second generation targeted therapy agents, and third generation immunotherapy agents. The first generation chemotherapy agents have severe side effects because they damage not only cancer cells but also normal cells. In other words, they attack normal cells to kill cancer cells, destroy the patient's immune system, and cause various side effects such as hair loss, vomiting, loss of appetite, fatigue, and extreme loss of physical strength due to strong toxicity. The second generation targeted therapy agents have the advantage of identifying and attacking only cancer cells, but they can only be used in patients with genetic mutations, making them impossible to treat various cancers and tend to develop resistance, so they cannot be used when resistance occurs. The third generation immunotherapy agents have a new mechanism of killing cancer cells by activating suppressed immune cells of the human body. They can be widely used for most cancers even without specific genetic mutations. Immunotherapy agents have fewer side effects in that they treat patients by strengthening the patient's own immunity, and have the effect of increasing the quality of life of cancer patients and significantly prolonging the survival period.

Immunotherapy agents exhibit anticancer effects by enhancing specificity, memory, and adaptiveness of the immune system. In other words, they use the body's immune system to accurately attack only cancer cells, resulting in fewer side effects. In addition, since immunotherapy agents use memory and adaptiveness of the immune system, a continuous anti-cancer effect can be seen in patients for whom immunotherapy agents are effective. Therefore, immunotherapy agents improved the side effects of the first generation chemotherapy agents and the resistance of the second generation targeted therapy agents, and are characterized by durable response, long-term survival, broad anti-tumor activity, and low toxicity profile.

Immunotherapy agents can be divided into passive immunotherapy agents and active immunotherapy agents. Passive immunotherapy agents include immune checkpoint inhibitors, immune cell therapy agents, and therapeutic antibodies. Immune checkpoint inhibitors are drugs that attack cancer cells by activating T cells by blocking the activation of immune checkpoint proteins involved in T cell suppression, and include CTLA-4, PD-1, and PD-L1 inhibitors. Immune cell therapy agents are drugs that enhance cellular immunity against cancer cells after collecting, strengthening and transforming T cells in the body through autologous cell transplantation (ACT) and injecting them, and include CAR-T cell therapy agents. In addition, therapeutic antibodies include antibody-drug conjugates (ADCs), etc., and when the antibody-drug conjugate binds to cancer cells, the drug is released and attacks cancer cells. Active immunotherapy agents include cancer treatment vaccines and immune-modulating agents. Cancer treatment vaccines are drugs that are made from cancer cells or substances produced by cancer cells and injected into the body to activate the body's natural defense system. In addition, immune-modulating agents are drugs that increase the immune response to cancer cells by activating the human immune response, such as activating specific white blood cells, and include cytokine therapy agents.

In anticancer immunotherapy using the immunotherapy agents, only patients with hypermutagenic MSI-H (about 20% of colorectal cancer patients) are considered as targets of anticancer immunotherapy that can expect a good prognosis, and patients with MSS (70˜80% of colorectal cancer patients) with an extremely small number of mutations compared to MSI-H do not benefit from anticancer immunotherapy.

Accordingly, in order to discriminate a patient group among MSS-type obese colorectal cancer patients to which anticancer immunotherapy can be applied, the present inventors studied a method for predicting the possibility of immunotherapy for a group of MSS-type obese colorectal cancer patients by using a discriminant of a multiple linear model for determining the applicability of anticancer immunotherapy to obese colorectal cancer patients, and completed the present invention.

### SUMMARY OF THE INVENTION

It is an object of the present invention to provide a method for predicting the possibility of immunotherapy for MSS-type colorectal cancer patients in order to determine a patient group to which anticancer immunotherapy can be applied among MSS-type obese colorectal cancer patients, which has recently rapidly increased.

To achieve the above object, the present invention provides an information providing method for predicting the possibility of immunotherapy for MSS-type colorectal cancer patients, comprising the following steps:

(a) a step of measuring the degree of gene mutation in MSS-type colorectal cancer patients;

(b) a step of classifying the patients into two groups according to the measured degree of gene mutation; and

(c) a step of determining the patient group with a high degree of the measured gene mutation as a group with high immunotherapy potential.

In addition, the present invention provides a system for predicting the possibility of immunotherapy for MSS-type colorectal cancer patients comprising the following units:

a measurement unit for measuring the degree of gene mutation in MSS-type colorectal cancer patients;

a classification unit for classifying the patients into two groups according to the measured degree of gene mutation;

and a determination unit for determining the patient group with a high degree of the measured gene mutation as a group with high immunotherapy potential.

### Advantageous Effect

The present invention relates to a method for predicting the possibility of immunotherapy for MSS-type colorectal cancer patients. Specifically, by using a discriminant of a multiple linear model for determining the applicability of anticancer immunotherapy to obese colorectal cancer patients of the present invention, the patients are classified into two patient groups according to the type of gene mutation, and a patient group with a high immune signature is selected as a group to which immunotherapy can be applied such that it is possible to provide the benefit of providing a new treatment opportunity to MSS-type colorectal cancer patients who have not benefited from the anticancer immunotherapy.

### BRIEF DESCRIPTION OF THE DRAWINGS

FIG. 1a shows the number of mutated genes comparing obese and normal weight subjects. FIG. 1b is a diagram showing the ratios of single nucleotide variants (SNV), frameshift insertions (INS) and deletions (DEL) in mutations in MSS-type obese colorectal cancer patients and normal weight colorectal cancer patients.

FIG. 2 is a diagram showing the degree of activation of an immune signature of MSS-type obese colorectal cancer patients compared to MSS-type normal weight colorectal cancer patients.

FIG. 3 is a diagram confirming the clusters of group 1 (G1) and group 2 (G2) with statistically significant differences by performing machine learning on the numbers of SNV and frameshift INDEL of MSS-type obese colorectal cancer patients.

FIG. 4 is a diagram comparing the activation levels of G1 and G2 immune signatures among MSS-type obese colorectal cancer patients.

FIG. 5 is a diagram showing the expression levels of CTLA4 and HAVCR2 in G1 among MSS-type obese colorectal cancer patients compared to MSS-type normal weight colorectal cancer patients.

### DESCRIPTION OF THE PREFERRED EMBODIMENTS

Hereinafter, the present invention is described in detail.

The present invention provides an information providing method for predicting the possibility of immunotherapy for MSS-type colorectal cancer patients, comprising the following steps:

(a) a step of measuring the degree of gene mutation in MSS-type colorectal cancer patients;

(b) a step of classifying the patients into two groups according to the measured degree of gene mutation; and

(c) a step of determining the patient group with a high degree of the measured gene mutation as a group with high immunotherapy potential.

Hereinafter, the information providing method for predicting the possibility of immunotherapy for MSS-type colorectal cancer patients of the present invention is described step by step in detail.

In the information providing method for predicting the possibility of immunotherapy for MSS-type colorectal cancer patients of the present invention, step (a) is a step of measuring the degree of gene mutation in MSS-type colorectal cancer patients.

The MSS-type colorectal cancer patient refers to a microsatellite stable (MSS) type colorectal cancer patient. The microsatellite refers to short repetitive DNA sequences scattered in chromosomes, and the microsatellite instability refers to a difference in length due to the insertion or deletion of repetitive sequences in the microsatellite within the cancer tissue when comparing the normal tissue and the cancer tissue in the same person. In other words, the microsatellite instability is a phenomenon in which the length of a repetitive microsatellite sequence distributed in all genes changes as the accumulation of point mutations of nucleotides accelerates because errors generated during the DNA replication process cannot be corrected due to abnormalities in the DNA mismatch repair system. When the gene repair system is damaged due to the microsatellite instability, tumors can develop while reducing the ability to relieve stress caused by chronic inflammation.

In the step (a), the degree of single nucleotide variant (SNV) and frameshift insertion and deletion (fsINDEL) in MSS-type colorectal cancer patients can be measured. Measuring the degree of gene mutation in MSS-type obese colorectal cancer patients can be to measure the degree of single nucleotide variant (SNV) and frameshift insertion and deletion (fsINDEL) in MSS-type colorectal cancer patients.

In addition, before the step (a), a step of selecting obese patients from among the MSS-type colorectal cancer patients can be further included.

In the step of selecting obese patients, an obese patient can be selected by measuring any one or more obesity-related indices selected from the group consisting of body mass index (BMI), waist-hip ratio (WHR), waist circumference (WC), waist-stature ratio (WSR), body fat percentage (BF %) and relative fat mass (RFM) of the patient, preferably selected by measuring body mass index (BMI) of the patient, but not always limited thereto.

The BMI index is a general obesity-related index for evaluating obesity, and is calculated by the following formula;

BMI=weight(kg)/[height(m)]²

Among the patients, in the case of Westerners, patients with a BMI of more than 30 can be classified as obese, patients with a BMI of more than 25 and less than 30 can be classified as overweight, and patients with a BMI index of less than 25 can be classified as normal weight. In the case of Asians, patients with a BMI of more than 25 can be classified as obese, patients with a BMI of more than 23 and less than 25 can be classified as overweight, and patients with a BMI of less than 23 can be classified as normal weight.

The WHR index is also known as the Kaufman index, and is an obesity-related index for evaluating abdominal obesity (visceral obesity or apple-shaped obesity), and is calculated by the following calculation formula;

WHR=waist circumference/hip circumference

Among the patients, in the case of men, patients with a WHR index of more than 0.95 can be classified as obese, and patients with a WHR index of less than 0.95 can be classified as normal weight. In the case of women, patients with a WHR index of more than 0.85 can be classified as obese, and patients with a WHR index of less than 0.85 can be classified as normal weight.

The WC index is an obesity-related index for measuring abdominal fat.

Among the patients, in the case of Western men, patients with a WC index of more than 94 cm can be classified as obese, and patients with a WC index of less than 94 cm can be classified as normal weight. In the case of Western women, patients with a WC index of more than 80 cm can be classified as obese, and patients with a WC index of less than 80 cm can be classified as normal weight. In addition, in the case of Asian men among the patients, patients with a WC index of more than 90 cm can be classified as obese, and patients with a WC index of less than 90 cm can be classified as normal weight. In the case of Asian women, patients with a WC index of more than 80 cm can be classified as obese, and patients with a WC index of less than 80 cm can be classified as normal weight.

The BF % index is an obesity-related index for measuring a patient's body fat percentage.

Among the patients, in the case of men, patients with a BF % index of more than 25% can be classified as obese, and patients with a BF % index of less than 25% can be classified as normal weight. In the case of women, patients with a BF % index of more than 32% can be classified as obese, and patients with a BF % index of less than 32% can be classified as normal weight.

The RFM index is an obesity-related index measured using a patient's waist circumference and height, and is calculated by the following calculation formula according to gender;

Female:RFM=76−(20×(height/waist circumference))

Male:RFM=64−(20×(height/waist circumference))

Among the patients, in the case of men, patients with an RFM index of more than 30% can be classified as obese, and patients with an RFM index of less than 30% can be classified as normal weight. In the case of women, patients with an RFM index of more than 40% can be classified as obese, and patients with an RFM index of less than 40% can be classified as normal weight.

In the information providing method for predicting the possibility of immunotherapy for MSS-type colorectal cancer patients of the present invention, step (b) is a step of classifying the patients into two groups according to the measured degree of gene mutation.

In the step (b), machine learning of dimensional conversion and clustering can be performed on the measured gene mutation values.

The dimensional conversion can be performed using any one of dimensional conversion technique selected from the group consisting of t-SNE (t-Stochastic Neighbor Embedding), PCA (Principal Component Analysis), LDA (Linear Discriminant Analysis), GDA (General Discriminant Analysis) and NMF (Non-negative Matrix Factorization), and t-SNE (t-Stochastic Neighbor Embedding) is preferably used, but not always limited thereto.

The clustering can be performed using any one of unsupervised learning technique selected from the group consisting of hierarchical clustering, k-means clustering, mixture model clustering, density-based spatial clustering of applications with noise (DBSCAN), generative adversarial networks (GAN) and self-organizing map (SOM), and k-means clustering is preferably used, but not always limited thereto.

The clustering can be performed using the following discriminant.

$${argmin}_{G} {\sum }_{i = 1}^{k} {\sum }_{x ∈ {G}_{i}} {‖ x - {μ}_{i} ‖}^{2} \qquad [ Discriminant 1 ]$$

In Discriminant 1 above, x may be an ordered pair (xSNV, xfsINDEL), xSNV may be the number of single nucleotide variants (SNV), xfsINDEL may be the number of frameshift insertions and deletions (fsINDEL), G is a set of patient groups in which the measured values of the mutation occurrence type of all patients are divided into k patient groups, which may be G={G1, G2, . . . , Gk}, and μi may be a centroid of observation values of the patients belonging to the patient group Gi.

The ‘COAD mutation dataset 2015-02-24’ version, which is the colorectal cancer patient gene mutation data used to derive the multiple linear model for discrimination of the Discriminant 1, can be downloaded from UCSC Cancer Genomics Browser. The ‘phenotype’ dataset of the ‘GDC TCGA Colon Cancer’ dataset can be downloaded from UCSC Xena Functional Genomics Explorer. For mutation data and gene expression data, ‘MuTect2 Variant Aggregation and Masking’, the ‘somatic mutation (SNPs and small INDELs)’ dataset of the ‘GDC TCGA Colon Cancer’ version, and ‘HTSeq—FPKM’, the ‘gene expression RNAseq’ dataset, respectively, can be downloaded from UCSC Xena Functional Genomics Explorer.

In one embodiment of the present invention, the MSS-type colorectal cancer patients could be specifically classified into group 1 (G1) and group 2 (G2) by machine learning on the number of nonsynonymous SNV (nsSNV) and fsINDEL using Discriminant 1 above. The number of nsSNV and fsINDEL of the patients of G1 may be statistically significantly higher than that of the patients of G2.

In the information providing method for predicting the possibility of immunotherapy for MSS-type colorectal cancer patients of the present invention, step (c) is a step of determining the patient group with a high degree of the measured gene mutation as a group with high immunotherapy potential.

In one embodiment of the present invention, among the MSS-type obese colorectal cancer patients, the G1 patient group having a higher number of nsSNV and fsINDEL compared to the G2 patient group could be determined as a group with high immunotherapy potential.

In addition, the present invention provides a system for predicting the possibility of immunotherapy for MSS-type colorectal cancer patients comprising the following units:

a measurement unit for measuring the degree of gene mutation in MSS-type colorectal cancer patients;

a classification unit for classifying the patients into two groups according to the measured degree of gene mutation; and

a determination unit for determining the patient group with a high degree of the measured gene mutation as a group with high immunotherapy potential.

The measurement unit can measure the degree of single nucleotide variant (SNV) and frameshift insertion and deletion (fsINDEL) of MSS-type colorectal cancer patients. Measuring the degree of gene mutation in MSS-type obese colorectal cancer patients can be to measure the degree of single nucleotide variant (SNV) and frameshift insertion and deletion (fsINDEL) in MSS-type colorectal cancer patients.

In addition, before the measurement unit, a selection unit for selecting obese patients from among the MSS-type colorectal cancer patients can be further included.

The selection unit can select an obese patient by measuring any one or more obesity-related indices selected from the group consisting of body mass index (BMI), waist-hip ratio (WHR), waist circumference (WC), waist-stature ratio (WSR), body fat percentage (BF %) and relative fat mass (RFM) of the patient, preferably select by measuring body mass index (BMI) of the patient, but not always limited thereto.

The BMI index is a general obesity-related index for evaluating obesity, and is calculated by the following formula;

BMI=weight(kg)/[height(m)]²

Among the patients, in the case of Westerners, patients with a BMI of more than 30 can be classified as obese, patients with a BMI of more than 25 and less than 30 can be classified as overweight, and patients with a BMI index of less than 25 can be classified as normal weight. In the case of Asians, patients with a BMI of more than 25 can be classified as obese, patients with a BMI of more than 23 and less than 25 can be classified as overweight, and patients with a BMI of less than 23 can be classified as normal weight.

The WHR index is also known as the Kaufman index, and is an obesity-related index for evaluating abdominal obesity (visceral obesity or apple-shaped obesity), and is calculated by the following calculation formula;

WHR=waist circumference/hip circumference

Among the patients, in the case of men, patients with a WHR index of more than 0.95 can be classified as obese, and patients with a WHR index of less than 0.95 can be classified as normal weight. In the case of women, patients with a WHR index of more than 0.85 can be classified as obese, and patients with a WHR index of less than 0.85 can be classified as normal weight.

The WC index is an obesity-related index for measuring abdominal fat.

Among the patients, in the case of Western men, patients with a WC index of more than 94 cm can be classified as obese, and patients with a WC index of less than 94 cm can be classified as normal weight. In the case of Western women, patients with a WC index of more than 80 cm can be classified as obese, and patients with a WC index of less than 80 cm can be classified as normal weight. In addition, in the case of Asian men among the patients, patients with a WC index of more than 90 cm can be classified as obese, and patients with a WC index of less than 90 cm can be classified as normal weight. In the case of Asian women, patients with a WC index of more than 80 cm can be classified as obese, and patients with a WC index of less than 80 cm can be classified as normal weight.

The BF % index is an obesity-related index for measuring a patient's body fat percentage.

Among the patients, in the case of men, patients with a BF % index of more than 25% can be classified as obese, and patients with a BF % index of less than 25% can be classified as normal weight. In the case of women, patients with a BF % index of more than 32% can be classified as obese, and patients with a BF % index of less than 32% can be classified as normal weight.

The RFM index is an obesity-related index measured using a patient's waist circumference and height, and is calculated by the following calculation formula according to gender;

Female:RFM=76−(20×(height/waist circumference))

Male:RFM=64−(20×(height/waist circumference))

Among the patients, in the case of men, patients with an RFM index of more than 30% can be classified as obese, and patients with an RFM index of less than 30% can be classified as normal weight. In the case of women, patients with an RFM index of more than 40% can be classified as obese, and patients with an RFM index of less than 40% can be classified as normal weight.

The classification unit can perform machine learning of dimensional conversion and clustering on the measured gene mutation values.

The dimensional conversion can be performed using any one of dimensional conversion technique selected from the group consisting of t-SNE (t-Stochastic Neighbor Embedding), PCA (Principal Component Analysis), LDA (Linear Discriminant Analysis), GDA (General Discriminant Analysis) and NMF (Non-negative Matrix Factorization), and t-SNE (t-Stochastic Neighbor Embedding) is preferably used, but not always limited thereto.

The clustering can be performed using any one of unsupervised learning technique selected from the group consisting of hierarchical clustering, k-means clustering, mixture model clustering, density-based spatial clustering of applications with noise (DBSCAN), generative adversarial networks (GAN) and self-organizing map (SOM), and k-means clustering is preferably used, but not always limited thereto.

The clustering can be performed using the following discriminant.

$${argmin}_{G} {\sum }_{i = 1}^{k} {\sum }_{x ∈ {G}_{i}} {‖ x - {μ}_{i} ‖}^{2} \qquad [ Discriminant 1 ]$$

In Discriminant 1 above,

x may be an ordered pair (xSNV, xfsINDEL), xSNV may be the number of single nucleotide variants (SNV), xfsINDEL may be the number of frameshift insertions and deletions (fsINDEL), G is a set of patient groups in which the measured values of the mutation occurrence type of all patients are divided into k patient groups, which may be G={G1, G2, Gk}, and μi may be a centroid of observation values of the patients belonging to the patient group Gi.

The ‘COAD mutation dataset 2015-02-24’ version, which is the colorectal cancer patient gene mutation data used to derive the multiple linear model for discrimination of the Discriminant 1, can be downloaded from UCSC Cancer Genomics Browser. The ‘phenotype’ dataset of the ‘GDC TCGA Colon Cancer’ dataset can be downloaded from UCSC Xena Functional Genomics Explorer. For mutation data and gene expression data, ‘MuTect2 Variant Aggregation and Masking’, the ‘somatic mutation (SNPs and small INDELs)’ dataset of the ‘GDC TOGA Colon Cancer’ version, and ‘HTSeq—FPKM’, the ‘gene expression RNAseq’ dataset, respectively, can be downloaded from UCSC Xena Functional Genomics Explorer.

In one embodiment of the present invention, the MSS-type colorectal cancer patients could be specifically classified into group 1 (G1) and group 2 (G2) by machine learning on the number of nonsynonymous SNV (nsSNV) and fsINDEL using Discriminant 1 above. The number of nsSNV and fsINDEL of the patients of G1 may be statistically significantly higher than that of the patients of G2.

In one embodiment of the present invention, among the MSS-type obese colorectal cancer patients, the G1 patient group having a higher number of nsSNV and fsINDEL compared to the G2 patient group could be determined as a group with high immunotherapy potential.

The MSS-type colorectal cancer patients may be MSS-type obese colorectal cancer patients or MSS-type normal weight colorectal cancer patients, but preferably, the MSS-type colorectal cancer patients may be MSS-type obese colorectal cancer patients.

Hereinafter, the present invention will be described in detail by the following examples.

However, the following examples are only for illustrating the present invention, and the contents of the present invention are not limited thereto.

### Example 1: Clustering of MSS-Type Colorectal Cancer Patients According to Obesity

Before analyzing the mutational properties of MSS-type colorectal cancer patients, clustering of MSS-type colorectal cancer patients was performed according to obesity in order to confirm the genetic difference between MSS-type obese colorectal cancer patients and MSS-type normal weight colorectal cancer patients.

Specifically, the body mass index (BMI) of MSS-type colorectal cancer patients was measured. Then patients with a BMI of 25 or more were classified as obese, and patients with a BMI of less than 25 were classified as normal weight. Among them, clustering was performed into two groups: a group of MSS-type obese colorectal cancer patients and a group of MSS-type normal weight colorectal cancer patients.

### Example 2: Comparison of Mutational Properties Between MSS-Type Obese Colorectal Cancer Patients and MSS-Type Normal Weight Colorectal Cancer Patients

In order to confirm the genetic difference between MSS-type obese colorectal cancer patients and MSS-type normal weight colorectal cancer patients, the mutational properties between the two patient groups were compared.

Specifically, the ratio of single nucleotide variant (SNV) and frameshift insertion (INS) and deletion (DEL) (fsINDEL) of each of MSS-type obese colorectal cancer patients and MSS-type normal weight colorectal cancer patients was statistically analyzed.

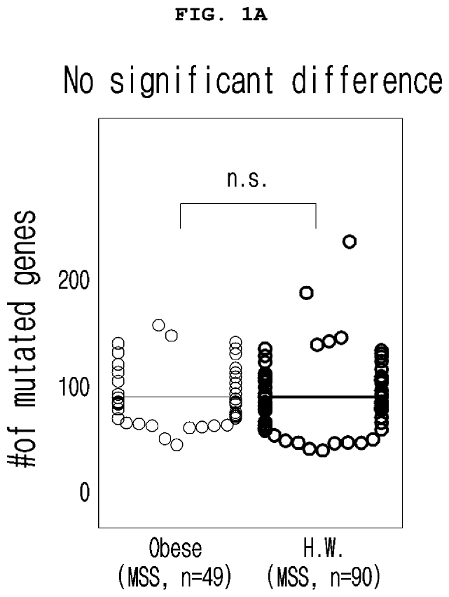

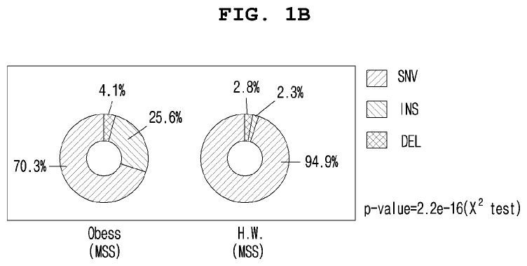

As a result, the overall numbers of mutated genes were not significantly different between obese and HW patients with MSS-CRC (FIG. 1a).

However, the MSS-type obese colorectal cancer patients showed a ratio of SNV of 70.3%, INS of 25.6%, and DEL of 4.1%, while the MSS-type normal weight colorectal cancer patients showed a ratio of SNV of 94.9%, INS of 2.3%, and DEL of 2.8%, so that insertions (INS) and deletions (DELs) were more common among obese patients than among HW patients (FIG. 1b).

### Example 3: Comparison of Immune Signatures Between MSS-Type Obese Colorectal Cancer Patients and MSS-Type Normal Weight Colorectal Cancer Patients

Since the higher the immune activity of MSS-type obese colorectal cancer patients, the higher the possibility of applying anticancer immunotherapy to the colorectal cancer patients, 8 immune signatures that can examine the immune status of MSS-type obese colorectal cancer patients were measured in comparison with MSS-type normal weight colorectal cancer patients.

Specifically, the degree of co-inhibition T cell activity, co-simulation APC activity, plasmacytoid dendritic cell (pDC) activity, co-inhibition APC activity, cytolytic activity, CD8+T cell activity, type II IFN response activity and MHC class I activity of MSS-type obese colorectal cancer patients was measured. The measurement was performed by the conventional immune signature measurement method (Cell; 2015 Jan 15;160(1-2):48-61).

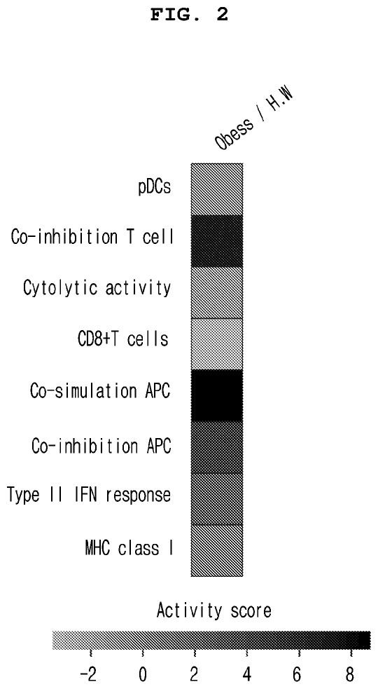

As a result, some immune signatures were activated in MSS-type obese colorectal cancer patients compared to MSS-type normal weight colorectal cancer patients, but CD8+T cell activity and cytolytic activity signatures, which are important in colorectal cancer immunotherapy, were observed to be lower in MSS-type obese colorectal cancer patients than in MSS-type normal weight colorectal cancer patients (FIG. 2).

### Example 4: Group Classification of MSS-Type Obese Colorectal Cancer Patients

### <4-1>Performing Dimensional Conversion on Mutation Data of MSS-Type Obese Colorectal Cancer Patients

Prior to clustering to determine the group of MSS-type obese colorectal cancer patients to which immunotherapy can be applied, dimensional conversion was performed on the mutation data of the MSS-type obese colorectal cancer patients of Example <3-2> for clustering.

Specifically, the number of nonsynonymous SNV (nsSNV) and fsINDEL that cause amino acid sequence mutations in MSS-type obese colorectal cancer patients was learned with a two-dimensional embedding vector that preserves the neighbor structure through dimensionality reduction using t-SNE (t-Stochastic Neighbor Embedding). The said t-SNE is one of the machine learning algorithms used for dimensionality reduction of data, and is a nonlinear dimensionality reduction technique useful for visualizing high-dimensional data by reducing it to two or three dimensions.

### <4-2>Derivation of Discriminant for Group Classification

In order to discriminate a group of MSS(microsatellite stability)-type obese colorectal cancer patients to which immunotherapy can be applied according to the degree of mutation, a discriminant of a multiple linear model for determining the applicability of anticancer immunotherapy to MSS-type obese colorectal cancer patients was derived.

Specifically, the multiple linear model for determining the applicability of anticancer immunotherapy to MSS-type obese colorectal cancer patients is composed of the counts of single nucleotide variant (SNV) and frameshift insertion and deletion (fsINDEL), and is as follows.

$${argmin}_{G} {\sum }_{i = 1}^{k} {\sum }_{x ∈ {G}_{i}} {‖ x - {μ}_{i} ‖}^{2}$$

(In the model above,

x is an ordered pair (xSNV, xfsINDEL), xSNV is the number of single nucleotide variants (SNV), xfsINDEL is the number of frameshift insertions and deletions (fsINDEL), G is a set of patient groups in which the measured values of the mutation occurrence type of all patients are divided into k patient groups, which is G={G1, G2, . . . , Gk}, and μi is a centroid of observation values of the patients belonging to the patient group Gi.)

The ‘COAD mutation dataset 2015-02-24’ version, which is the colorectal cancer patient gene mutation data used to derive the multiple linear model for discrimination, was downloaded from UCSC Cancer Genomics Browser1. The ‘phenotype’ dataset of the ‘GDC TCGA Colon Cancer’ dataset can be downloaded from UCSC Xena Functional Genomics Explorer2. For mutation data and gene expression data, ‘MuTect2 Variant Aggregation and Masking’, the ‘somatic mutation (SNPs and small INDELs)’ dataset of the ‘GDC TCGA Colon Cancer’ version, and ‘HTSeq—FPKM’, the ‘gene expression RNAseq’ dataset, respectively, were downloaded from UCSC Xena Functional Genomics Explorer.

### <4-3>Performing Group Classification of MSS-Type Obese Colorectal Cancer Patients

In order to discriminate a group of MSS-type obese colorectal cancer patients to which anticancer immunotherapy can be applied according to the degree of gene mutation, MSS-type obese colorectal cancer patients were classified into two groups.

Specifically, k-means clustering was performed using the discriminant of Example <4-2> based on the gene mutation data of MSS-type obese colorectal cancer patients performed dimensionality reduction in Example <4-1>. K-means clustering is an algorithm that groups given data into k clusters, and is a machine learning that operates in a way that minimizes the variance of each cluster and distance difference.

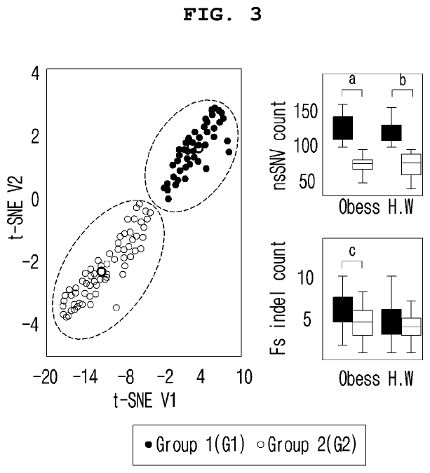

As a result of clustering, it was confirmed that the number of SNV and fsINDEL was divided into two clusters with significant differences. [symbol] , Therefore, in MSS-type obese colorectal cancer patients, the cluster with high SNV and fsINDEL numbers was named Group 1 (G1), and the cluster with low SNV and fsINDEL numbers was named Group 2 (G2) (FIG. 3).

### Example 5: Confirmation of Immune Signature and Immune Checkpoint Gene Expression Levels by Group Among MSS-Type Obese Colorectal Cancer Patients

In order to confirm a group of MSS-type obese colorectal cancer patients to which anticancer immunotherapy can be applied, 8 immune signatures of the two groups G1 and G2 classified in Example 4 were analyzed. The immune signature analysis was performed in the same manner as in Example 3.

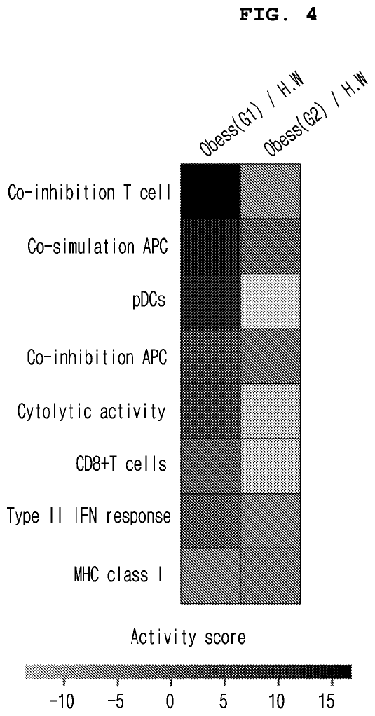

As a result, among MSS-type obese colorectal cancer patients, the patients belonging to G1 were confirmed to have 7 more activated immune signatures (Co-inhibition T cell, Co-simulation APC, pDCs, Co-inhibition APC, Cytolytic activity, CD8+ T cells and Type II IFN response) compared to MSS-type normal weight colorectal cancer patients. In particular, it was also confirmed that CD8+T cell and cytolytic activity signatures, which are important for anticancer immunotherapy, were more activated in the patients belonging to G1 than in MSS-type normal weight colorectal cancer patients (FIG. 4).

On the other hand, among MSS-type obese colorectal cancer patients, the patients belonging to G2 showed lower or similar activity in 8 immune signatures compared to MSS-type normal weight colorectal cancer patients.

These results suggest that among MSS-type obese colorectal cancer patients, the patients belonging to G1 have higher immune activity compared to the patients belonging to G2, indicating that anticancer immunotherapy is highly likely to be applied to the patients belonging to G1.

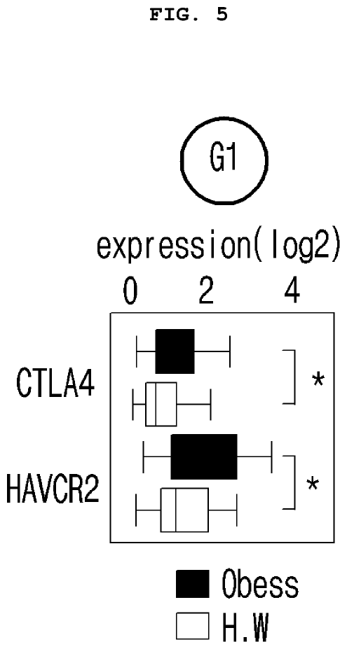

In addition, if the activity of CD8+ T cells is high, they can attack tumor cells (or cancer cells) well, and if the cytolytic activity is high, the expression level of immune checkpoint genes is high, so immune cells can be induced to attack tumor cells (or cancer cells) through immune checkpoint suppression (PNAS, 1998 Mar. 17;95(6):3111-3116, Clin cancer Res. 2017 Jun. 15; 23(12):3129-3138). Accordingly, the expression levels of immune checkpoint genes in the MSS-type obese colorectal cancer patients belonging to G1 and the MSS-type normal weight colorectal cancer patients belonging to G1 with the same criteria were examined. As a result, it was confirmed that CTLA4 and HAVCR2 expression levels in the MSS obese colorectal cancer patients belonging to G1 were significantly higher than those in normal weight patients (FIG. 5).

CTLA4 is a target of immunotherapy drugs for MSI—H type colorectal cancer patients approved by the US FDA, and is significantly different in G1 obese patients with. Considering the high expression levels of immune checkpoint genes (CTLA4 and HAVCR2) in MSS-type obese colorectal cancer patients, it can be predicted that anticancer immunotherapy has the potential to act on the group of MSS-type obese colorectal cancer patients.

### INDUSTRIAL APPLICABILITY

The patients are classified into two patient groups according to the type of gene mutation by using a discriminant of a multiple linear model for determining the applicability of anticancer immunotherapy to obese colorectal cancer patients of the present invention, and a patient group with a high immune signature is selected as a group to which immunotherapy can be applied such that it is possible to provide the benefit of providing a new treatment opportunity to obese colorectal cancer patients who have not benefited from the anticancer immunotherapy.

What is claimed is:

1. An information providing method for predicting the possibility of immunotherapy for MSS-type colorectal cancer patients, comprising the following steps:
(a) a step of measuring the degree of gene mutation in MSS-type colorectal cancer patients;
(b) a step of classifying the patients into two groups according to the measured degree of gene mutation; and
(c) a step of determining the patient group with a high degree of the measured gene mutation as a group with high immunotherapy potential;
wherein a step of selecting obese patients from among the MSS-type colorectal cancer patients is further included before the step (a)
wherein the step (a) is to measure the degree of single nucleotide variant (SNV) and frameshift insertion and deletion (fsINDEL) in MSS-type colorectal cancer patients.

2. The information providing method for predicting the possibility of immunotherapy for MSS-type colorectal cancer patients according to claim 1, wherein the obese patient is selected by measuring any one or more obesity-related indices selected from the group consisting of body mass index (BMI), waist-hip ratio (WHR), waist circumference (WC), waist-stature ratio (WSR), body fat percentage (BF %) and relative fat mass (RFM) of the patient in the step of selecting obese patients.

3. The information providing method for predicting the possibility of immunotherapy for MSS-type colorectal cancer patients according to claim 1, wherein the step (b) is to perform dimensional conversion and clustering on the measured gene mutation values.

4. The information providing method for predicting the possibility of immunotherapy for MSS-type colorectal cancer patients according to claim 3, wherein the dimensional conversion is performed using any one of dimensional conversion technique selected from the group consisting of t-SNE (t-Stochastic Neighbor Embedding), PCA (Principal Component Analysis), LDA (Linear Discriminant Analysis), GDA (General Discriminant Analysis) and NMF (Non-negative Matrix Factorization).

5. The information providing method for predicting the possibility of immunotherapy for MSS-type colorectal cancer patients according to claim 3, wherein the clustering is performed using any one of unsupervised learning technique selected from the group consisting of hierarchical clustering, k-means clustering, mixture model clustering, density-based spatial clustering of applications with noise (DBSCAN), generative adversarial networks (GAN) and self-organizing map (SOM).

6. The information providing method for predicting the possibility of immunotherapy for MSS-type colorectal cancer patients according to claim 3, wherein the clustering is performed using the following discriminant.
$${argmin}_{G} {\sum }_{i = 1}^{k} {\sum }_{x ∈ {G}_{i}} {‖ x - {μ}_{i} ‖}^{2} \qquad [ Discriminant 1 ]$$
(In Discriminant 1 above,
x is an ordered pair (xSNV, xfsINDEL), xSNV is the number of single nucleotide variants (SNV), xfsINDEL is the number of frameshift insertions and deletions (fsINDEL), G is a set of patient groups in which the measured values of the mutation occurrence type of all patients are divided into k patient groups, which is G={G1, G2, . . . , Gk}, and μi is a centroid of observation values of the patients belonging to the patient group Gi.)

7. A system for predicting the possibility of immunotherapy for MSS-type colorectal cancer patients comprising the following units:
a selection unit for selecting obese patients from among the MSS-type colorectal cancer patients;
a measurement unit for measuring the degree of gene mutation in MSS-type colorectal cancer patients;
a classification unit for classifying the patients into two groups according to the measured degree of gene mutation; and
a determination unit for determining the patient group with a high degree of the measured gene mutation as a group with high immunotherapy potential;
wherein the measurement unit measures the degree of single nucleotide variant (SNV) and frameshift insertion and deletion (fsINDEL) in MSS-type colorectal cancer patients.

8. The system for predicting the possibility of immunotherapy for MSS-type colorectal cancer patients according to claim 7, wherein the selection unit selects an obese patient by measuring any one or more obesity-related indices selected from the group consisting of body mass index (BMI), waist-hip ratio (WHR), waist circumference (WC), waist-stature ratio (WSR), body fat percentage (BF %) and relative fat mass (RFM) of the patient.

9. The system for predicting the possibility of immunotherapy for MSS-type colorectal cancer patients according to claim 7, wherein the classification unit performs dimensional conversion and clustering on the measured gene mutation values.

10. The system for predicting the possibility of immunotherapy for MSS-type colorectal cancer patients according to claim 9, wherein the dimensional conversion is performed using any one of dimensional conversion technique selected from the group consisting of t-SNE (t-Stochastic Neighbor Embedding), PCA (Principal Component Analysis), LDA (Linear Discriminant Analysis), GDA (General Discriminant Analysis) and NMF (Non-negative Matrix Factorization).

11. The system for predicting the possibility of immunotherapy for MSS-type colorectal cancer patients according to claim 9, wherein the clustering is performed using any one of unsupervised learning technique selected from the group consisting of hierarchical clustering, k-means clustering, mixture model clustering, density-based spatial clustering of applications with noise (DBSCAN), generative adversarial networks (GAN) and self-organizing map (SOM).

12. The system for predicting the possibility of immunotherapy for MSS-type colorectal cancer patients according to claim 9, wherein the clustering is performed using the following discriminant.
$${argmin}_{G} {\sum }_{i = 1}^{k} {\sum }_{x ∈ {G}_{i}} {‖ x - {μ}_{i} ‖}^{2} \qquad [ Discriminant 1 ]$$
(In Discriminant 1 above,
x is an ordered pair (xSNV, xfsINDEL), xSNV is the number of single nucleotide variants (SNV), xfsINDEL is the number of frameshift insertions and deletions (fsINDEL), G is a set of patient groups in which the measured values of the mutation occurrence type of all patients are divided into k patient groups, which is G={G1, G2, . . . , Gk}, and μi is a centroid of observation values of the patients belonging to the patient group Gi.)
